(19) United States
(12) Patent Application Publication
(10) Pub. No.: US 2006/0241563 A1
(43) Pub. Date: Oct. 26, 2006

(54) Adjustable safety clip

(75) Inventors: Michael Vogel, Karlsruhe (DE); Jurgen Dorn, Neulussheim (DE)

Correspondence Address: MORRISON & FOERSTER, LLP, 555 WEST FIFTH STREET, SUITE 3500, LOS ANGELES, CA, 90013-1024, US

(73) Assignee: C. R. Bard, Inc., Murray Hill, NJ (US)

(21) Appl. No.: 10/564,341

(22) PCT Filed: Jul. 23, 2004
(86) PCT No.: PCT/EP04/08288

§ 371 Date: Jan. 24, 2006

(30) Foreign Application Priority Data
Jul. 24, 2003 (GB) 0317359.8

(51) Int. Cl. A61M 25/00

(52) U.S. Cl. 604/523

(57) ABSTRACT

A safety clip (100) for a medical catheter features a slider (6) for stepless adjustment of a distance separating an abutment surface for a sleeve (20) of the catheter from an abutment surface for a shaft (22) of the catheter.

The present invention relates to a safety clip for a medical catheter device. More particularly, the invention relates to a safety clip for maintaining the position of the end of a medical catheter relative to the position of the end of either a push rod or pull wire. For an example of such a clip, see FIG. 9 of WO 98/10713.

There are many fields of medical surgery which utilize medical instruments comprising a catheter to be inserted into the various passageways of the human body. The catheter is then advanced through the passageways of the human body, for example through blood vessels or the biliary tree, until the distal end of the catheter reaches the desired treatment side.

Depending on the type of surgery to be performed, the medical catheter device will normally consist of one or more concentric, tubular members which can be moved in a length wise direction proximally or distally relative to one another. This is usually achieved by actuation of an appropriate mechanism at the proximal end of the medical catheter device. The result of this actuation at the distal end might be to deploy a medical device such as a stent at the treatment side, or to operate some medical tool at the distal end of the catheter, to manipulate bodily tissue or a prosthesis.

Normally, a medical catheter device will reach the surgeon packaged in a condition ready for use, such that little or no assembly or preparation of the device is necessary on the part of the surgeon before commencing a procedure.

In the interest of patient safety, the medical catheter device should not be actuated until the surgeon has positioned the distal end of the catheter at the correct, predetermined site within the body.

Therefore, to ensure that the device arrives in a condition where it can be used immediately, and in order that the device is not unintentionally operated during insertion of the catheter into, and guidance of the catheter through, the body passageways, it is known to provide a safety clip at the proximal end of the device in order to prevent premature or inadvertent movement of the catheter and other concentric tubular members, relative to one another, at the distal end of the device.

A typical medical catheter device will comprise a catheter with either a push rod or a pull wire within a lumen of the catheter which are moved relative one another at the proximal end. The safety clip is thus positioned adjacent to the proximal ends of the catheter and push rod or pull wire to prevent their relative motion.

In a medical catheter device, for example a percutaneous intraluminal stent delivery catheter, there are a range of diameters of suitable catheters and other tubular members. Similarly, the length of stent that must be delivered will vary according to the patient and according to the needs of the medical condition. Further, likely will be slight differences in length of the various catheters and other tubular members arising during manufacture of the medical catheter devices, due to machine tolerances, human inolvement, etc.

In order to solve these and other problems, there is provided a safety clip for a medical catheter as claimed in claim 1 below. The clip can maintain the spacing between the ends of a push rod or pull wire and the catheter, and can comprise: an elongate, resilient body, having a first end, a second end and an intermediate portion; attachment means for attaching the safety clip to at least one of the push rod or pull wire and the catheter; and a first spacing member at the first end of the elongate body, and characterized in that a distance between the first spacing member and the attachment means can be adjusted.

According to a second aspect of the present invention, there is provided a medical device comprising: a catheter; a push rod or pull wire; and a safety clip, attached to said medical device, for maintaining the spacing between the ends of said catheter and said push rod or pull wire, characterized in that the safety clip has means for adjusting the distance between its ends in order to achieve the desired fit for the particular medical catheter to which it is attached, whereby the distance between the ends of the safety clip may be fixed when said safety clip is attached to said medical device.

In the case of a catheter and push rod combination, the safety clip is designed to fit between the proximal ends of the two members, in order to prevent depression of the push rod relative to the catheter. In the case of a pull wire, the safety clip performs in tension to prevent the retraction of the pull wire relative to the catheter.

In either case, the attachment member may attach the safety clip either to the catheter or to the push rod or pull wire. It is conceived that the attachment may also be made between the clip and part of a housing or other constructional feature, which is itself directly connected to one of the tubular members, rather than to the member itself.

A spacing member then abuts the other tubular member, or its housing or a stopper connected to the tubular member, to prevent the undesired depression of the push rod or retraction of the pull wire. It is further conceived that the spacing member may be designed to engage the tubular members in a manner that prevents both retraction and depression, either by providing a stopper either side of the spacing member, or by providing a spacing member that lies either side of a stopper, for instance.

The present invention also encompasses having an attachment means approximately centrally located along the length of the safety clip, with a spacing member at either end of the elongate body. Thus, the attachment means grips the relevant component, whilst the spacing members prevent unwanted relative motion of the tubular members by abutting the appropriate structures to prevent relative sliding motion between the tubular members.

In order to achieve the required adjustment in spacing between the various spacing members and the attachment means, it is conceived that any or all of the attachment means, first spacing member and second spacing member may be adjustable. Alternatively, or in addition to this, the elongate body itself may have an adjustable length.

Once the required length has been set, the spacing of the safety clip can be fixed, either by a locking mechanism, such as a ratchet device or engagement button, by the adjustment being modular, e. g. by pegging of the various components into suitable holes along the length of the elongate body, or by application of a suitable adhesive to prevent further movement of a sliding or rotating component.

In this manner, it is possible to provide an adjustable safety clip which can be used on a range of devices, and each individual clip then adjusted to fit the size of the specific medical catheter device to which it is applied. This ensures that any variations due to manufacturing tolerances, varying requirements of surgery and devices for a differing range of procedures can all be accommodated by a single design of clip.

Further, it is considered that the attachment means should be suitable for attaching the device to a range of catheters and push rods or pull wires, such that it can accommodate a variation in diameter of the tubular member to which it is attached. One way to achieve this is to provide the attachment means with a series of jaws or engagement means of varying diameter, within a range appropriate to the medical devices to which it is to be attached. Thus, a different portion of the attachment means will contact the device, depending on the specific diameter of the device being used.

Further advantage can be gained by providing the attachment means, in the region to be attached to the tubular member, with a component made of a suitable thermoplastic, such that when the thermoplastic is heat treated, it will soften and conform to the shape of the surface to which it is attached, thereby creating a superior engagement between the safety clip and medical catheter device.

In order that the invention may be more easily understood, and by way of example, reference will now be made to the drawings, in which:

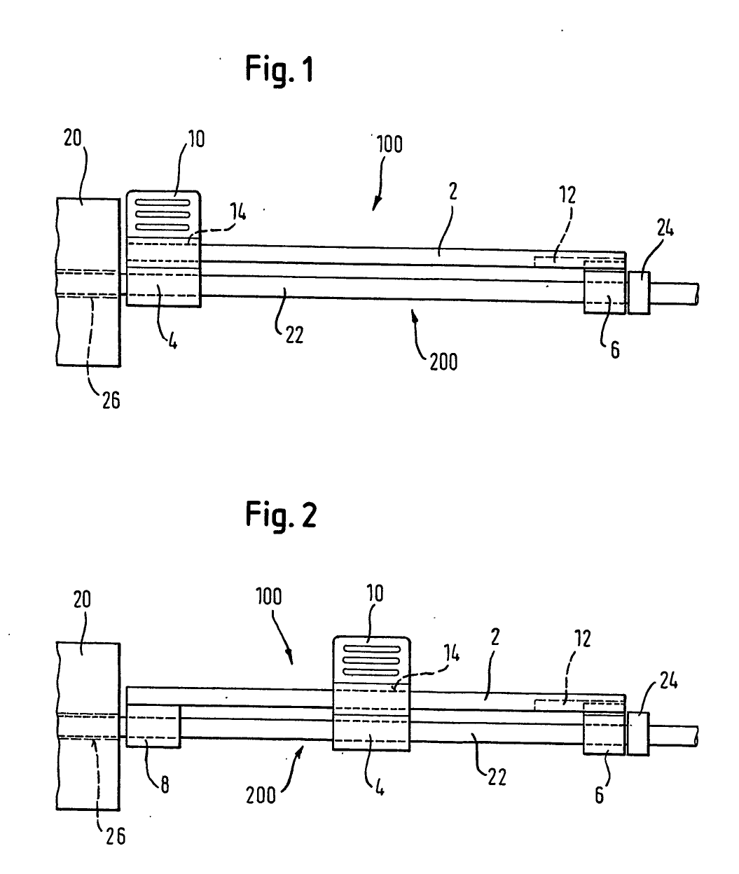

FIG. 1 is a side view of a safety clip attached to a medical catheter device, according to a first embodiment;

FIG. 2 is a side view of a safety clip attached to a medical catheter device, according to a second embodiment;

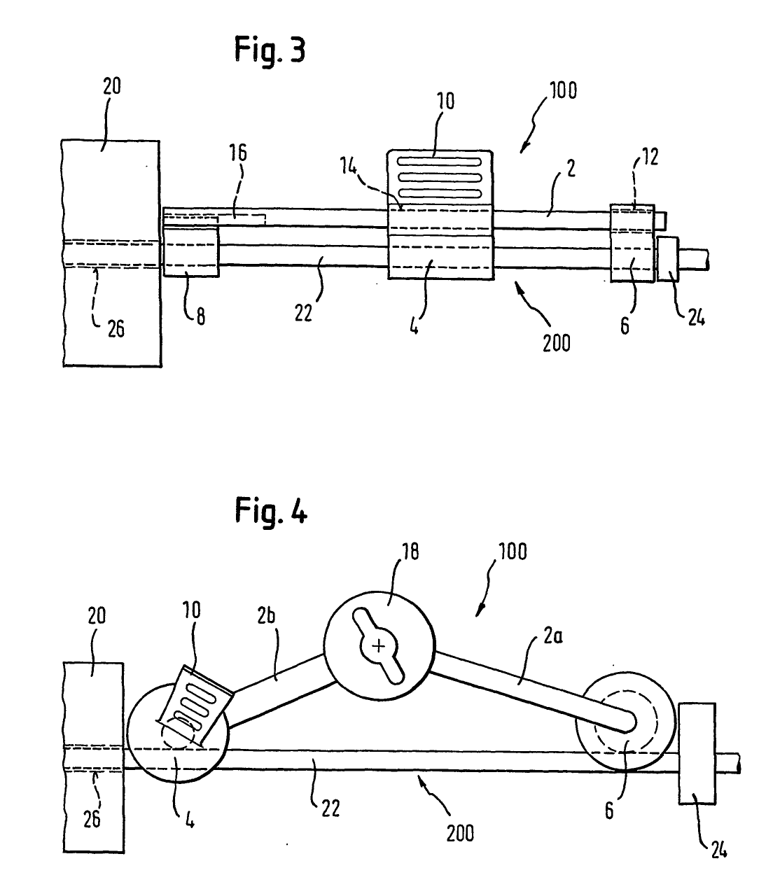

FIG. 3 is a side view of a safety clip attached to a medical catheter device, according to a third embodiment;

FIG. 4 is a side view of a safety clip attached to a medical catheter device, according to a fourth embodiment;

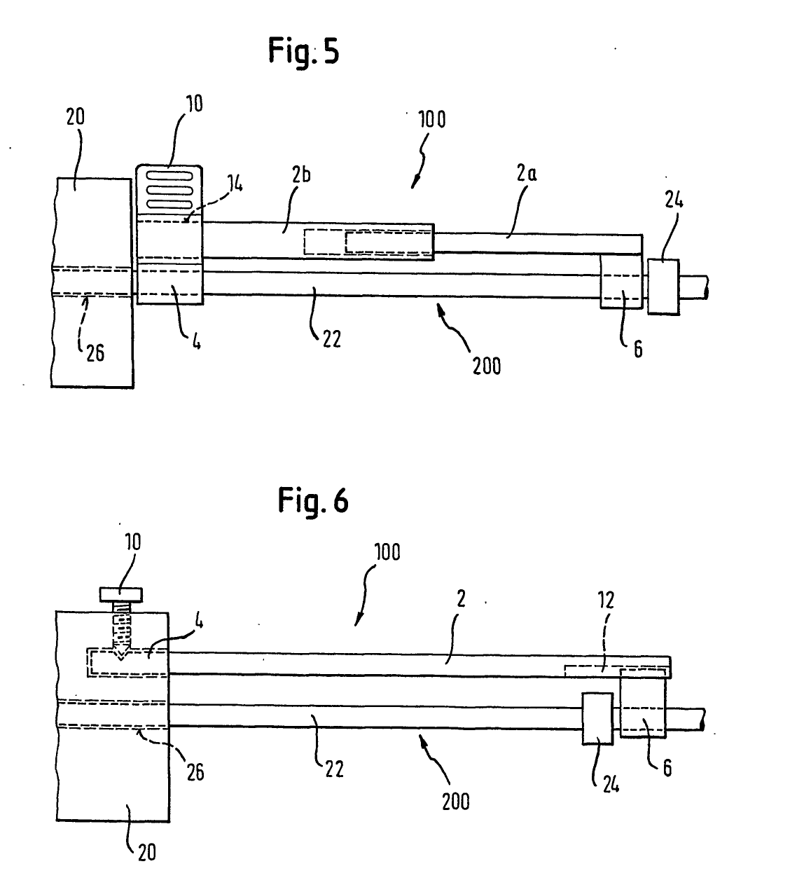

FIG. 5 is a side view of a safety clip attached to a medical catheter device, according to a fifth embodiment;

FIG. 6 is a side view of a safety clip attached to a medical catheter device, according to a sixth embodiment;

FIG. 7 is a perspective view of one embodiment of an attachment means;

FIG. 8 is a cross-sectional view of an attachment means, according to a further embodiment;

FIG. 9 is a cross-sectional view of a spacing member, according to one embodiment;

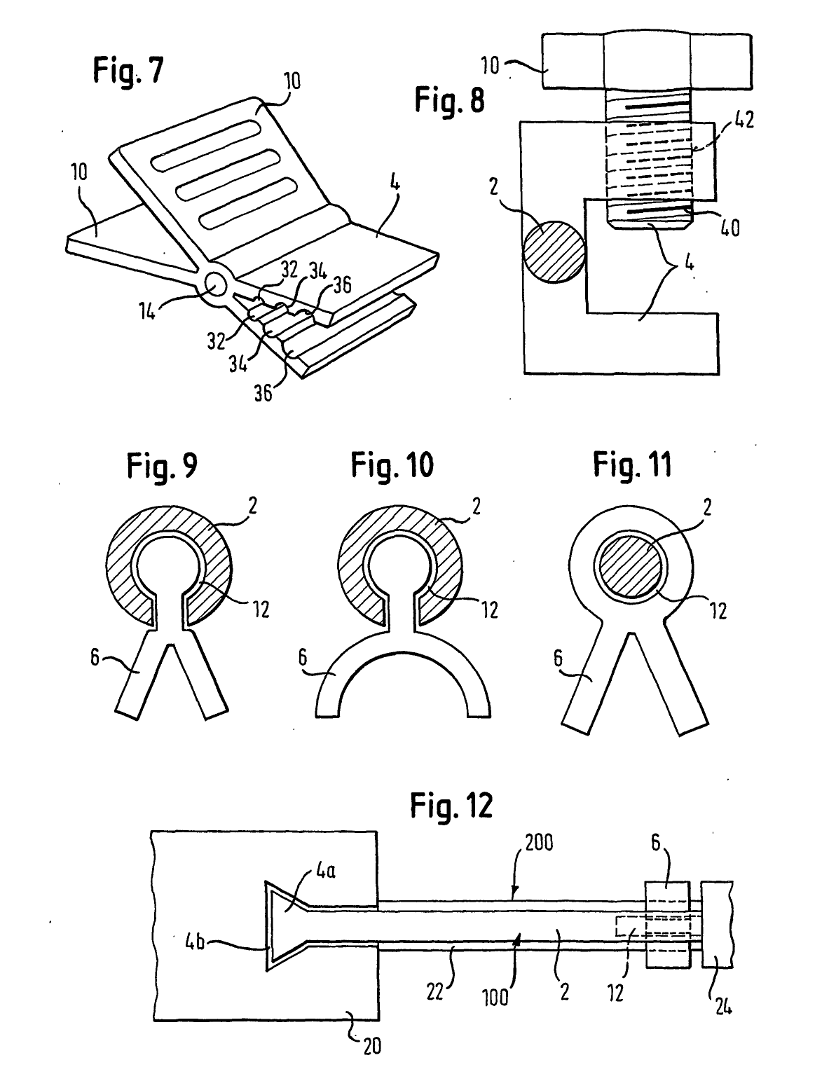

FIG. 10 is a cross-sectional view of a spacing member, according to a second embodiment;

FIG. 11 is a cross-sectional view of a spacing member, according to a third embodiment;

FIG. 12 is a plan view of a safety clip, showing an alternative means of attaching the safety clip to the medical catheter device.

Throughout the Figures, the same reference numerals have been used to refer to similar, identical or corresponding features.

FIG. 1 shows a schematic side view of a safety clip 100 comprising elongate, semi-rigid body 2, attachment means 4 and first spacing member 6. The position of the first spacing member 6 can be adjusted by sliding the first spacing member longitudinally along the lengthwise slot 12 at the first end of the elongate body. The attachment means is provided with a releasing means 10 for releasing the attachment means from the medical catheter device 200. Optionally, the longitudinal position of the attachment means 4 may be made adjustable, by the provision of a bore 14 through a connecting portion of the attachment means to allow the attachment means to slide longitudinally along the elongate body.

In the Figure, the safety clip 100 is shown in combination with a medical catheter device 200 comprising a catheter or catheter housing 20 and push rod 22. The catheter and push rod can slide longitudinally relative one another, the push rod 22 being slidable within the lumen or bore 26 through the catheter 20. The push rod is provided with a stopping member 24 which abuts the first spacing member 6, whilst the attachment means 4 abuts the catheter 20. Because the specific distance between the stopping member 24 and catheter 20 may vary, depending on the type of medical catheter device being used, and due to variation between medical catheter devices of the same type, the distance between attachment means 4 and the first spacing member 6 is adjustable in order to take up any slack and to ensure a correct fit of the safety clip into the gap between the stopping member and the catheter.

In order to correctly apply the safety clip, the end of the safety clip adjacent to the catheter is positioned abutting the catheter whilst the spacing member 6 is adjusted to abut the stopping member 24. The position of the spacing member 6 is then fixed at the correct distance. This may be done by providing a locking means, such as an engagement button, or by providing a ratchet surface allowing travel in only one direction for the first spacing member 6 within the slot 12. Alternatively, the first spacing member may be glued in position. Rather than providing a slot 12, there could be a series of holes along the length of the elongate body, with matching peg or pegs on the spacing member, such that the position of the spacing member can be chosen, in a modular fashion, by pegging the spacing member into the correct holes. A combination of stepped and stepless length adjustment may be particularly beneficial, when precise length-setting over a large length range is needed.

In the case where the attachment means also has an adjustable position, its position must also be fixed in some similar, suitable manner.

In the schematic embodiment shown, the attachment means 4 and releasing means 10 form a single body similar to a clothes peg, whereby release of the safety clip from the medical catheter device is achieved by squeezing the lever arms 10 to release the gripping jaws 4 from the push rod 22. The spacing member in this case comprises a V-shaped member, such that the push rod fits within the notch of the V, with the apex towards the elongate body. In this instance, there is no gripping action between the spacing member 6 and the push rod 22.

The safety clip shown schematically in FIG. 2 is similar to that shown in FIG. 1, except that the attachment means 4 is located within the intermediate portion of the elongate body 2, and there is a second spacing member 8 located at the second end of the elongate body. The second spacing member 8 is positioned to abut the catheter 20, whilst the attaching means 4 maintains a connection with the push rod 22. The first spacing member 6 is adjustable to ensure that it abuts the stopping member 24. Again, the attachment means 4 may be in a fixed position relative to the elongate body 2, or may be allowed to move relative to the elongate body by an optional bore 14.

The embodiment shown schematically in FIG. 3 is similar to that of FIG. 2, except that the second spacing member 8 can also move longitudinally relative to the elongate body 2 along a lengthwise slot 16. As an illustrative example, in this embodiment, the first spacing member 6, instead of being slidable along a slot 12, is instead provided with a bore 12 through which the elongate body 2 may pass, in order to allow the first spacing member to slide longitudinally along the elongate member.

FIG. 4 shows a safety clip 100 which has an attachment means 4 at the second end of the elongate body 2, and a first spacing member 6 at the first end of the elongate body 2. In this embodiment, the elongate body consists of two sections 2a and 2b which are hinged by a rotating hinge member 18. The hinging member 18 can be locked in position, for example by the provision of a circular ratchet, or by a clamping means. In this manner, the distance between attaching means 4 and the first spacing member 6 can be adjusted by rotation of the hinge member 18 in order to vary the angle between the two sections 2a and 2b of the elongate body. Again, releasing means 10 is also provided so that attachment means 4 can be released from the push rod 22. The design of the attachment means 4 and first spacing member 6 is such that the clip is able to grip and maintain contact with the push rod 22 over a range of angles between the two portions 2a and 2b of the elongate body.

The embodiment according to FIG. 5 is substantially the same as the embodiment according to FIG. 1, except that, rather than the first spacing member 6 being movable relative to the elongate body 2, the elongate body instead comprises two portions 2a and 2b which are longitudinally adjustable. In this illustrative example, the elongation is permitted by thinner tubular member 2a sliding within a bore provided within larger tubular member 2b. Thus, the distance between the attachment means 4 and the first spacing means 6 can be adjusted.

In FIG. 6, an embodiment is disclosed this time illustrated for a medical catheter device 200 which has a pull wire 22, rather than a push rod. Hence, stopper 24 is located on a different side of the first spacing member 6. In this embodiment, the attachment means 4 comprises a system which functions in combination with the catheter housing 20 in order to attach the safety clip 100 to the catheter housing 20, rather than to the pull wire 22. There is provided a releasing means 10 which ordinarily engages the attachment means 4 in order to secure the safety clip 100 in position. The releasing means 10 is removed in order to release the safety clip from the medical catheter device.

FIG. 7 shows a possible embodiment of an attachment means 4, in combination with a releasing means 10. The device functions as a gripping means, similar to a clothes peg, wherein attaching means 4 is ordinarily configured to grip a push rod or pull wire 22 within one of the sets of jaws 32, 34, 36. Squeezing the lever members 10 together causes the jaws to open, thus releasing the tubular member held within. If the attachment means is not formed as a fixed part of the safety clip, through-bore 14 may also be provided in order to allow the attachment means to slide longitudinally along the elongate body.

In a preferred embodiment, the material of the jaws 32, 34 and 36 is made from a thermoplastic. The different jaws then have different curvatures, suitable for different diameters of push rod or pull wire. However, for intermediate sizes the thermoplastic can then be heat treated to become soft, such that the jaws may be pressed against a push rod or pull wire of a diameter different to the initial size of the jaws, in order that the jaws then conform to the specific diameter of the push rod or pull wire in use.

FIG. 8 shows a possible embodiment of an attachment means, in this case a screw-clamping device, provided somewhere along the length of the elongate body 2. Releasing means 10 functions to turn the threaded member 40 within a threaded bore 42, thus closing the attachment means 4. Releasing means 10 can then be twisted in the reverse direction, prior to use of the medical catheter device, in order to release the attachment means from the catheter, or push rod or pull wire, to which it is attached.

FIGS. 9, 10 and 11 show possible cross-sectional arrangements for the spacing member 6 or 8 and elongate body 2. In FIGS. 9 and 10 the elongate body 2 is provided with a lengthwise slot 12 or 16 along which the spacing member 6 or 8 can slide. In FIG. 11, the spacing member 6 or 8 is provided with a through-bore 12 or 16 so that it can slide along the elongate body 2.

FIG. 12 is a plan view showing a safety clip 100 provided with dove-tail section 4a attached to a catheter housing 20 with dove-tail slot 4b, such that the combination provides an attachment mechanism between the catheter housing 20 and safety clip 100. First spacing member 6 is then provided at the first end of the elongate body 2, and can slide along lengthwise slot 12, as in the previous embodiments. The safety clip 100 can be removed simply by lifting the safety clip out of the dove-tail slot provided in the catheter body.

According to the above embodiments, there is provided an adjustable safety clip for use with medical catheter devices which can provide several advantages, some of which are mentioned below.

Because the safety clip is able to accommodate a range of diameters of catheter and push rod or pull wire, the same safety clip is able to be used on a wide range of medical devices. Similarly, because the safety clip can be applied to a range of differing types of medical catheter device, for use in a range of surgical procedures, the same safety clip can be used throughout many fields of surgery. As well as making the safety clip versatile, this also lends itself to mass-production of the safety clip.

Because the safety clip is correctly adjusted to fit the catheter device to which it is connected, the medical device itself will arrive, packaged, in a condition ready for a surgeon to perform a medical procedure, without having to prime or re-set the medical catheter device prior to insertion. Similarly, the surgeon is able to perform the operation with increased confidence that no danger will arise to the patient due to inadvertent operation of the medical catheter device function, during insertion of the device through the body passageways.

Because the adjustable safety clip can be produced with only a single moving part, the entire device can be fabricated from a suitable, e. g. plastics, material in a one-piece molding with a further molded spacing member or attachment means. As such, the number of components, and hence the manufacturing complexity, is reduced.

Similarly, if the single molding is made from a suitable thermoplastic, the attachment means can be heat treated to achieve an improved fit with the component to which it is attached. Therefore, reliability is improved that the clip will not become unintentionally dislodged from the medical catheter device. Also, this reduces the impact of manufacturing or molding deficiencies on the performance of the safety clip, since defects may be overcome during the heat treating process.

Adjustable safety clips of this type can be mass produced for use as disposable safety clips. This is particularly relevant to modern surgical apparatus, where it is often economically more viable to use disposable equipment, rather than to forgo the expense of maintaining the equipment sterile.

A further advantage may be achieved that the safety clip is easily removable from the medical catheter device, ensuring that the medical catheter device is not disturbed during removal of the safety clip. Such disturbance could, for example, dislodge the distal end of the medical catheter device causing the device to move away from the desired treatment side within the human body. With the present invention, it is possible to make the safety clip detachable in such a way that little or no force is exerted on the medical catheter device itself, ensuring that the medical catheter device remains undisturbed.

1. A safety clip (100) for a medical catheter that comprises a shaft (22) within a sleeve (20), the clip serving to maintain the respective proximal ends of the rod and the sleeve against a relative axial movement, the clip comprising:
an elongate body (2), having a first end, a second end and an intermediate portion;
attachment means (4) for attaching the safety clip to the catheter; and
a sleeve abutment surface at one end of the body and a shaft abutment surface at the other end of the body, the body being effective to maintain spaced apart said respective abutment surfaces, and characterized by
a slider (4, 6, 8) for stepless adjustment of the distance separating said sleeve abutment surface and said shaft abutment surface.

2. The safety clip according to claim 1, wherein the attachment means (4) defines one of the sleeve abutment surface and the shaft abutment surface.

3. The safety clip according to claim 1 or 2, wherein the attachment means (4) is slidable on the body.

4. The safety clip according to claim 1 or 3, wherein the attachment means (4) is spaced from both ends of the elongate body.

5. The safety clip according to any preceding claim, wherein the slider (4, 6, 8) slides on the body (2) and defines one of the sleeve abutment surface and the shaft abutment surface.

6. The safety clip according to any preceding claim, wherein the slider (18) comprises a hinged member.

7. The safety clip according to any preceding claim, wherein at least one of the sleeve abutment surface and the shaft abutment surface is defined by a pair of jaws with a jaw opening adapted to accommodate a range of diameters of the shaft or sleeve, respectively.

8. The safety clip according to any preceding claim, further comprising releasing means (10) for releasing the attachment means (4) from the catheter, without disturbing the spacing between said abutment surfaces.

9. A safety clip as claimed in any one of the preceding claims, attached to a medical catheter.

10. A safety clip as claimed in any one of the preceding claims, in which the distance between said abutment surfaces has been selected, and fixed against any further adjustment.
